(19) United States
(12) Patent Application Publication
(10) Pub. No.: US 2011/0312022 A1
(43) Pub. Date: Dec. 22, 2011

(54) METHOD FOR DETERMINING PERCENT HYBRIDITY IN MODIFIED HYBRID CROPS

(75) Inventor: Danielson B. Gardner, Wilton, CA (US)

(73) Assignee: AGRIGENETICS, INC., Indianapolis, IN (US)

(21) Appl. No.: 12/879,041

(22) Filed: Sep. 10, 2010

Related U.S. Application Data
(60) Provisional application No. 61/243,568, filed on Sep. 18, 2009.

(51) Int. Cl. C12Q 1/06

(52) U.S. Cl. 435/39

(57) ABSTRACT

Methods of introducing sentinel traits into foundation seed are described. The frequency of occurrence of the sentinel trait in the hybrid seed product relative to the frequency of the sentinel trait in a pollinator seed product are used to determine the percentage of hybridity in modified hybrid varieties.

### CROSS-REFERENCE TO RELATED APPLICATIONS

This application claims the benefit of U.S. Provisional Application No. 61/243,568, filed on Sep. 18, 2009 which is expressly incorporated by reference herein.

### FIELD OF THE INVENTION

This invention is in the field of plant breeding. In particular, the invention relates to methods for determining the percentage of hybridity in a seed product.

### BACKGROUND

Hybrid plant varieties are superior to traditional open pollinated plant varieties. Hybrid plant variety production has revolutionized plant breeding practices for improving plant species. Enhancements such as increased yield, plant uniformity, rate of development, fruitfulness, and disease resistance are a few examples of improved characteristics that are possible in hybrid seed.

In the United States, there are two types of hybrids allowed under the Federal Seed Act; at least 95% hybrid seed, and at least 75% hybrid seed. 7 CFR §201.26 (2005). Most greater than 95% hybrid seed is produced using alternating female and male rows in the field and harvesting the seed from just the female rows to be sold as hybrid. Modified hybrids (greater than 75% hybrid) can include in addition to the hybrid seed, seed born from self pollinations of the male line. Methods for determining the percentage of hybrid seed contained within a bag of seed labeled and marketed as “hybrid seed” are needed to insure the percentage of hybrid seed solids at least 75% hybrid seed.

Currently, several methods are used to test the percentage of hybridity in a seed lot. These include; grow-out tests of the seed to evaluate phenotypic markers, inspection of production fields, molecular markers and isozyme markers. These tests require additional expense and time. Improved solutions which provide more efficient methods for verifying the actual percentage of hybrid seed are desirable.

This invention provides a solution for identifying the percentage of male inbred seed in a harvested population of modified hybrid seed. The method disclosed can be used to calculate the percentage of hybridity in a hybrid seed sample.

### SUMMARY

In one embodiment, the invention provides a method for determining the percentage of male inbred seed in a hybrid seed product. Female plants (from at least one female line) are grown from seed and crossed with pollinator plants (from at least one pollinator line) grown from pollinator seed. An additional pollinator line that expresses the sentinel trait is added to the pollinator seed to produce mixed pollinator seed. Plants from the female seed and the mixed pollinator seed are grown under conditions that result in (i) crossing of plants from the mixed pollinator seed and plants from the female seed to produce the hybrid seed product and (ii) selfing and/or sibbing of plants from the mixed pollinator seed to produce a pollinator seed product. The occurrence of the sentinel trait in the hybrid seed product and in the pollinator seed product is determined. The percentage of pollinator product in the hybrid seed product is determined from the occurrence of the sentinel trait in the hybrid seed product and in the pollinator seed product.

In another embodiment, the invention provides a method for determining the percentage of hybridity in a seed product. Female line plants (from at least one female line) are grown from female seed and crossed with pollinator line plants (from at least one pollinator line) grown from pollinator seed. An additional pollinator line that expresses the sentinel trait is added to the pollinator seed to produce mixed pollinator seed. Plants of the mixed pollinator line and plants of the female line are grown under conditions that result in crossing of the pollinator line plants and the female line plants to produce the hybrid seed product. Plants of the pollinator line are grown under conditions that result in selfing and or sibbing of the pollinator line plants to produce a pollinator seed product. The occurrence of the sentinel trait in the hybrid seed product and in the pollinator seed product is determined and used to calculate the percentage of hybridity of the hybrid seed product.

Other aspects of the invention will become apparent by consideration of the detailed description.

### DETAILED DESCRIPTION

The present invention relates to a method for determining the percentage of hybrid seed in a hybrid seed product. As used herein, the term “hybrid seed” refers to seed produced on female plants as a result of being crossed to different pollinator plants. Suitably, the seed product contains at least about 70%, at least about 75%, at least about 80%, at least about 85%, at least about 90%, at least about 95%, at least about 97%, or at least about 99% of hybrid seed.

As used herein the term “seed product” refers to a seed product that includes hybrid seed produced on female plants crossed with pollinator plants as well as selfed and/or sibbed and/or crossed seed from the pollinator plants. The present invention has applicability to a broad variety of hybrid seed. In particular, the present invention may be used for any hybrid seed that may be produced from an incompletely controlled pollination, such as hybrid seed that is produced from a blend of seed, e.g., a uniform lot of seed containing seed of female plants and seed of pollinator plants. Suitable plants in which methods of the invention may be used include, without limitation, alfalfa, soybean, canola, sunflower, maize, orchard grass, as well as cereals such as oat, wheat, barley, rice, rye and triticale.

In another embodiment the invention relates to growing a collection of isolated pollinator plants in mixture with sentinel trait plants in pollinating proximity to produce the pollinator seed product. As used herein, the term “pollinator plants” refers to male fertile plants used to pollinate female plants in order to produce the resulting hybrid seed. The principal function of the pollinator plant is fertilization of the female plant by shedding ample amounts of pollen within a field.

As used herein, the term “isolated” refers to pollinator plants that are spatially separated from female plants as to be harvested independent from the female plants.

As used herein, a “sentinel trait” is a heritable phenotypic trait that can be measured, evaluated or observed in a plant or seed without genetic analysis, such as DNA sequencing or molecular marker analysis, of the plant or seed. A sentinel trait contained in a seed may, for example, be evident in the seed, and/or in the plant grown from the seed. For example, the sentinel trait may be evaluated by visual inspection of the seed or plant, or may be determined using tools, equipment or machinery that measures the trait.

Depending on the characteristics of the sentinel trait, testing or evaluation of the sentinel trait may be carried out on seeds of the hybrid seed product and of the pollinator seed product, or on plants grown from seeds of the hybrid seed product and pollinator seed product. For example, the sentinel trait may be a recessive phenotypic trait in the male parent. Suitable sentinel traits include, without limitation, seed texture, seed coat luster (e.g., dull, shiny), seed coat hair density, seed coat hair length, seed coat color (e.g., white seed coat, black seed coat, yellow seed coat), seed size, seed weight, embryo composition (e.g. starch content, waxy type), seed density, two unifoliate leaves, multi-foliate leaves, multiple cotyledons, glandular hairs, fall dormancy and flower color. Sentinel traits producing a phenotype in the seed, such as seed coat color or seed size, can be evaluated using the seeds themselves. Other traits that are expressed in the plant as phenotypes such as two unifoliate leaves, multi-foliate leaves, multiple cotyledons, and flower color may be detected and scored in plants grown from the seed.

In another embodiment, the invention relates to growing female plants and pollinator plants comprising a sentinel trait in close proximity (e.g., pollinating proximity) to produce the hybrid seed product. As used herein, the term “female plants” refers to plants where pollen is removed or not produced. Female plants may be emasculated via methods known in the art, including classical breeding, mechanical emasculation, or chemical emasculation to develop female plants. The female plants may be produced from a cross of male sterile and maintainer plants. It is understood that female plants may produce a small amount of pollen.

In another embodiment, the invention relates to determining a percentage of seed displaying the sentinel trait in the hybrid seed product and separately determining a percentage of seed displaying the sentinel trait in the pollinator seed product. Suitably, the hybrid seed product contains at least about 70%, at least about 75%, at least about 80%, at least about 85%, at least about 90%, at least about 95%, at least about 97%, or at least about 99% of hybrid seed.

As used herein, “pollinator seed product” refers to the seed obtained from selfed and/or sibbed and/or crossed pollinator plants. The pollinator seed product is suitably of the same generation as the pollinator component of the hybrid seed product. The plants producing the pollinator seed product may be grown under substantially similar conditions and/or substantially the same time as the plants producing the hybrid seed product. For example, the pollinator seed product may be obtained from pollinator plants grown or isolated at the edge of a production field in which a hybrid seed product is produced, or along side or in proximity to plants grown to produce the hybrid seed product.

In another embodiment, the invention relates to determining the percentage of hybridity of the seed product according to the formula:

$$\% Hybridity = ( \frac{\begin{matrix}1 - \% SENTINEL TRAIT \\ IN HYBRID SEED PRODUCT\end{matrix}}{\begin{matrix}\% SENTINEL TRAIT IN \\ POLLINATOR SEED PRODUCT\end{matrix}} ) \times 100. \qquad Equation 1$$

Verification or estimation of the amount of hybrid seed, hybridity level, or percentage of hybridity in a hybrid seed product may be determined by testing or evaluating the hybrid seed product and pollinator seed product and scoring a sentinel trait. The invention provides methods of facilitating the production of hybrid seed crops by introducing into the pollinator product the presence of one or more sentinel traits and the monitoring of the sentinel trait in the hybrid seed product. Production fields of a given plant variety may be planted such that the pollinator and female plants produce at least 75% hybrid seed in a hybrid seed product.

The hybrid seed product is suitably produced from a first parent of one or more female plants and a second parent of one or more pollinator plants. Tests or evaluation of the hybrid seed product and the pollinator seed product may be carried out when the plants or seed have phenotypic sentinel traits that can be scored. In one embodiment, the sentinel trait is present in the pollinator parent but not in the female parent. The percent hybridity of the hybrid seed product may be calculated using the frequency of the sentinel trait in the hybrid seed product (or plants grown from the hybrid seed product) and the frequency of the sentinel trait in the pollinator seed product (or plants grown from the pollinator seed product) according to the Equation 1.

In hybridized crops, the female parent plant may be produced by, for example and without limitation, crossing a female line to a maintainer plant. This step may be suitably performed in the field in isolation by planting the female plants and the maintainer line plants in separate rows and harvesting the seed born on the female plants separately from the seed born on the maintainer plants. The resulting male sterile seed harvested from the female plants produces female seed which may be crossed with a pollinator line to produce the hybrid seed product. The pollinator line can include an inbred line, a single cross hybrid, or a population of any type. Seed produced by female plants from pollinations by pollinator plants is hybrid seed. Seed produced from selfed or sibbed pollinator plants is non-hybrid seed.

The pollinator line and sentinel pollinator line are mixed at a pre-determined ratio. Plants of the male line and sentinel pollinator line are allowed to cross pollinate, self pollinate and sib mate in isolation from other plants. The pollinator seed product harvested from the isolated pollinator and sentinel pollinator plants may be tested for the sentinel trait, and the frequency of the trait in the pollinator seed product determined The frequency of the sentinel trait in the pollinator component of the hybrid seed product may be determined using the frequency of the sentinel trait in the pollinator seed product that is of the same generation as would be recovered in the hybrid seed product. Suitably, the isolation of the pollinator plants may be done by growing the pollinator line in or proximal to a production field in which the hybrid seed product is produced. Suitably, the pollinator seed product is produced from plants grown under substantially similar conditions as the plants used to produce the hybrid seed product.

When the sentinel trait is a recessive trait, such as the white seed coat in alfalfa, present in the pollinator line and not present in the female line, hybrid seed from the cross of the female plants with the pollinator plants do not express the sentinel trait. The hybrid seed product, which includes hybrid seed and non-hybrid seed, may be tested to determine the frequency of the sentinel trait in the hybrid seed product. Hybridity of the hybrid seed product may then be calculated. The quotient of the frequency of the sentinel trait in the hybrid seed product divided by the frequency of the sentinel trait in the pollinator seed product may be determined to provide a measure of the hybridity of the hybrid seed product. In particular, one minus the quotient of the frequency of the sentinel trait in the hybrid seed product divided by the frequency of the sentinel trait in the pollinator seed product and multiplied by one hundred may be used to provide an estimate of the percent hybridity in the hybrid seed product.

The present invention further provides a method for introducing a sentinel trait to a pollinator line without backcrossing. In this embodiment, seed from a second pollinator line carrying a sentinel trait (sentinel trait seed) is suitably added to the pollinator parent. The second pollinator line having a sentinel trait is herein referred to as the sentinel trait parent and the mixture of seed from the sentinel trait parent and from pollinator parent is herein referred to as mixed pollinator seed. The amount of Sentinel pollinator to add to the pollinator line parent is added such that its presence has minimal effect on the integrity of the variety, but is sufficiently high to be scored effectively. Suitably, the Sentinel pollinator line seed is added in an amount of at least about 0.000001%, at least about 0.00001%, at least about 0.0001%, at least about 0.001%, at least about 0.01%, at least about 0.1%, at least about 0.5% or at least about 1% by weight of the Pollinator line seed. Suitably, the sentinel pollinator line seed is added in an amount of less than about 10%, less than about 9%, less than about 8%, less than about 7%, less than about 6%, less than about 5%, less than about 4%, less than about 3% or less than about 2% by weight of the Pollinator line seed.

Adding sentinel trait seed to the pollinator seed to produce mixed pollinator seed and allowing plants grown from the mixed pollinator seed to self or sib in one generation to produce a pollinator seed product, facilitates the establishment of a seed yield ratio of pollinator parent to sentinel trait parent. The pollinator seed product (or plants grown from the pollinator seed product) from the selfed and/or sibbed and/or crossed mixed pollinator plants may be tested or evaluated for the sentinel trait, and the frequency of the trait in the pollinator seed product may be determined.

Plants from the mixed pollinator seed are suitably grown in isolation, for example, in or along side of a hybrid production field. Plants grown from the mixed pollinator seed are also suitably used to pollinate female plants to produce a hybrid seed product. The frequency of the sentinel trait in the pollinator seed product may then be used to establish the percentage of pollinator seed product in the hybrid seed product, as shown above in Equation 1.

The frequency of the sentinel trait may be determined in the pollinator seed product and hybrid seed product or in plants grown from the pollinator seed product and hybrid seed product. The sentinel trait is suitably distinguished in the seeds or progeny of the sentinel trait parent from the seeds or progeny from the pollinator parent and the hybrid seeds or hybrid progeny from the male-sterile parent.

The seed yield of the female line (hybrid seed) and the seed yield of the pollinator parent are suitably not altered significantly by combining the sentinel trait parent with the pollinator parent. For example, the hybrid seed yield from the female line pollinated by the mixed pollinator plants is at least about 95%, at least about 97%, at least about 98%, or at least about 99% of the hybrid seed yield from the female line pollinated by the pollinator line, and less than about 105%, less than about 103%, less than about 102% or less than about 101% of the hybrid seed yield from the female line pollinated by the pollinator line, when grown under substantially similar conditions.

In one embodiment, the relative seed yield of the female seed parent and the pollinator parent is established for a given planting ratio of female parent to pollinator parent and may be used to approximate the proportion or percentage of hybridity in a hybrid variety grown at that planting ratio. Planting ratios of the female parent and pollinator parent may be adjusted to maximize seed yield or hybridity of the hybrid variety, and may be determined, for example, by evaluating the expression ration of male sterility to male fertility in the field. Suitably the planting ratio of female parent and pollinator parent is at least about 50:50, at least about 60:40, at least about 70:30 or at least about 80:20. Suitably the planting ratio of female parent and pollinator parent is less than about 95:5, is less than about 90:10, or is less than about 85:15.

The sentinel trait parent may suitably express the sentinel trait fully, or less than fully. For example the sentinel trait may be expressed in the sentinel trait parent at least about 85%, at least about 90%, at least about 95%, at least about 97%, at least about 98% or at least about 99% expression. High expression, of the sentinel trait may result in more accurate analysis and may facilitate a lower ratio of sentinel trait parent to pollinator being required. However, sentinel traits expressed at low frequencies may also be used. Suitably, the sentinel trait is expressed in at least about 1%, at least about 5%, at least about 15%, at least about 30%, or at least about 50% of the sentinel trait seeds and/or sentinel trait plants. The invention further provides methods for evaluating the optimum planting ratio of female line to pollinator line of new hybrids that do not contain phenotypic markers, have not had molecular markers determined, and/or are early in varietal development. Early evaluation of female to pollinator ratios facilitates an estimation of a hybrid's seed yield potential and the minimization of seed production costs while still maintaining hybridity standards. Each female line will have a slightly different yield potential with different pollinator lines. By using a mixed pollinator parent, the optimum planting ratio for each hybrid parent combination may be determined.

The optimal planting ratio of a female line to pollinator line for producing sufficient seed at a sufficiently high percentage of hybridity may be deduced by determining the relative seed yield of the female line and pollinator line.

The determination of the percentage of sentinel trait in hybrid seed product is based on the percentage of sentinel trait in a sample taken from the harvested seed product. When the sentinel trait is expressed at low frequencies, such as where the sentinel trait is expressed at or below 1%, the size of the sample taken from the harvested seed product will preferably be large enough to represent a similar percentage of sentinel trait as in the harvested field. One having ordinary skill in the art would understand that conventional statistical methods such as a statistical sample size calculation could readily be used to determine an appropriate sample size based on sentinel trait expression and other variables.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are illustrative and are not to be construed as limiting the scope of the invention.

### EXAMPLE 1

### Use of a White-Seeded Recessive Trait as a Sentinel Trait in Production of Hybrid Alfalfa

Hybrid alfalfa was produced by crossing a female line (male sterile) with a mixed pollinator line in the ratio of 65% female and 35% pollinator. The mixed pollinator seeds were comprised of about 96% yellow seeded types and 4% white seeded (sentinel trait) types. The white seed coat color displayed by the sentinel pollinator seeds is a recessive phenotypic trait. The hybrid seed produced from the cross of the female line with the mixed pollinator line did not express the white seed coat color sentinel trait. A mixture of female seed and mixed pollinator line seed in the ratio of about 65:35 was planted in a production field in Fresno County, California. A plot was planted with only mixed pollinator seed within the production field. The plot containing seed product from the selfing and/or sibbing and/or crossed seed from the mixed pollinator plants was harvested first. Then the remaining production field was harvested as a hybrid seed product containing a mixture of hybrid seed born from the female plants and non-hybrid seed from the mixed pollinator line plants. The harvested seeds from the production field and the isolated plot were each cleaned and conditioned in an identical manner, and samples of the lots were scored for the frequency of white seed. Five samples from the hybrid totaling about 70,000 seeds and five samples from the pollinator plot totaling about 15,000 seeds were scored for the sentinel trait.

The frequency of white seed in a sample of the hybrid seed product was determined to be 0.25846% and the frequency of the white seed in the pollinator was determined to be 1.05314%. The percent hybridity in the hybrid sample was determined using this data. The percent hybridity in the hybrid product was calculated as being equal to 1 minus the quotient of the percent white seed in the hybrid sample divided by the percent white seed in the male check, according to the following equation:

$$\% Hybridity = ( \frac{1 - \% WHITE SEED IN HYBRID SEED PRODUCT}{\% WHITE SEED IN POLLINATOR SEED PRODUCT} ) \times 100$$

The hybrid seed product was calculated to have 75.46% hybridity.

### EXAMPLE 2

### Use of a Yellow-Seeded Recessive Trait as a Sentinel Trait in Production of Black-Seeded Hybrid Canola (Brassica napus)

A hybrid seed field is planted at 75% black seeded female and with a 25% mixed pollinator line that was 96% black seeded and 4% yellow seeded. A small portion of the field is planted with only the mixed pollinator. The hybrid and mixed pollinator line portions of the field are harvested separately, the percentage yellow seed in each lot of seed is determined, and the percentage hybridity is calculated, according to the following equation:

$$\% Hybridity = ( \frac{1 - \% YELLOW SEED IN HYBRID SEED PRODUCT}{\% YELLOW SEED IN POLLINATOR SEED PRODUCT} ) \times 100$$

A percent hybridity of at least 75% is expected.

### EXAMPLE 3

### Use of a Waxy-Mutant Single-Gene Recessive Trait as a Sentinel Trait in Production of Hybrid Rice

A seed field is planted at 75% normal female and 25% with a mixed pollinator that consisted of 96% normal pollinator line and 4% waxy seeded sentinel line. A portion of the field is planted with only the mixed pollinator. The hybrid and pollinator line portions of the field are harvested separately, and the percent % waxy seed in each lot of seed is determined. Seeds with the waxy mutation are distinguished visually, or by treatment with iodine, which stains the waxy mutants brown and the normal types blue. The percentage hybridity is calculated, according to the following equation:

$$\% Hybridity = ( \frac{1 - \% WAXY SEED IN HYBRID SEED PRODUCT}{\% WAXY SEED IN POLLINATOR SEED PRODUCT} ) \times 100$$

A percent hybridity of at least 75% is expected.

What is claimed is:

1. A method for determining the percentage of hybridity in a seed product, the method comprising the steps of:
(a) growing a collection of isolated pollinator plants comprising a sentinel trait in pollinating proximity;
(b) growing female plants and pollinator plants comprising a sentinel trait in pollinating proximity to produce the seed product;
(c) determining a percentage of seed displaying the sentinel trait in the hybrid seed product and separately determining a percentage of seed displaying the sentinel trait in the seed on the pollinator seed product; and
(d) determining the percentage of hybridity of the seed product according to the formula:
$$\% Hybridity = ( \frac{1 - \% SENTINEL TRAIT IN HYBRID SEED PRODUCT}{\% SENTINEL TRAIT IN POLLINATOR SEED PRODUCT} ) \times 100$$

2. The method of claim 1, wherein the pollinator seed having a sentinel trait is present in the mixed pollinator seed in an amount from about 1% to about 6%.

3. The method of claim 1, wherein the sentinel trait comprises a recessive trait.

4. The method of claim 1, wherein the sentinel trait is seed color.

5. The method of claim 1, wherein the hybrid seed product comprises alfalfa seed.

6. The method of claim 5, wherein the sentinel trait comprises a white seed coat.

7. The method of claim 1, wherein the sentinel trait comprises a maternally inherited trait.

8. The method of claim 1, wherein the percentage of hybridity is at least seventy-five percent.

9. The method of claim 1, wherein the plants grown from the mixed pollinator seed and the plants grown from the female seed to produce the hybrid seed product are crossed at a planting ratio of about 60:40 to 80:20.

10. The method of claim 1, wherein the occurrence of the sentinel trait is determined by evaluating intact seeds of the hybrid seed product and the pollinator seed product.

11. The method of claim 1, wherein the occurrence of the sentinel trait is determined by evaluating intact plants grown from seed of the hybrid seed product and from plants of the pollinator seed product.
